(19) United States
(12) Patent Application Publication
(10) Pub. No.: US 2023/0028230 A1
(43) Pub. Date: Jan. 26, 2023

(54) SYSTEM AND METHOD FOR BIO-MEDICAL SAMPLES IDENTIFICATION AND RESOURCE OPTIMIZATION

(71) Applicant: Rfxlabs India Pvt Ltd, Mumbai (IN)

(72) Inventor: Sunil Mahabaleshwar Faide, Mumbai (IN)

(73) Assignee: Rfxlabs India Pvt Ltd, Mumbai (IN)

(21) Appl. No.: 17/493,848

(22) Filed: Oct. 5, 2021

(30) Foreign Application Priority Data
Jul. 26, 2021 (IN) 202121033593

(51) Int. Cl. G16H 10/65; G16H 40/60

(52) U.S. Cl. CPC G16H 10/65; G16H 40/60

(57) ABSTRACT

The system (100) and method for bio-medical samples (07) identification and resource optimization, as compared to prior-arts, is capable to continuously read and record, without manual-intervene, multiple bio-medical sample holders, individually/in groups and provides effective recording, communication and resource management of bio-medical sample holders and resource management at a processing point. System (100) has an outgoing mail pick-up bin (40) at a collection point (10) that continuously scans multiple RFID tags (32) of the bio-medical sample tagged holders (30) dropped therein at an instant to achieve digital data and by use of the RFID communication device (44) connected to a cloud network (20) intimates a processing point (50) to plan for resources optimization before bio-medical arrival of bio-medical samples and the location of dispatch. The collection point (10) does not have to manually scan and record and hence increases efficiency.

### FIELD OF THE INVENTION

The present disclosure relates to a system and method for bio-medical samples identification and resource optimization at a collection point to keep account of bio-medical samples while handling and avoid misplacing, misidentification, facilitating record management, facilitating quick turnaround of bio-medical samples and managing and replenishment of resources with minimal manpower. Particularly, the present disclosure relates to a system and method for bio-medical samples identification and resource optimization at a collection point that in real-time is capable to continuously scan and record multiple bio-medical samples, individually or in groups and assist in resource optimization.

### BACKGROUND OF THE INVENTION

Collection of bio-medical samples of different patients or persons undergoing bio-medical examination is everyday task at a collection point which can be a clinic, a hospital or a residential place like old age homes in which a doctor or a skilled person collects bio-medical samples. It is known that the number of bio-medical samples collected at such collection point is large in numbers and it is difficult to manually label sample holders, record and dispatch bio-medical samples to one or more processing points, namely laboratories, for processing and examination of collected bio-medical samples. To name a few, bio-medical samples can be biopsies (pieces of tissue removed surgically) or other samples from a person or a patient including samples of blood, urine, stool, scrapings from the skin, nose, throat or any other body locations, hair etc.

In the known process, the bio-medical samples are collected and positioned in respective known bio-medical sample holders and then manually labeled with the name of patient or patient reference number. At times more than one bio-medical sample holders are placed in one or more biohazard bags and forwarded to one ore more processing points. The collection point manually makes a list of patients with details like the name of the examination to be conducted for each patient and other necessary details like age, gender and date and time of collection and shares the information to the processing point via fax, email or a written document which is sent with bio-hazard bags. Upon arrival of the bio-medical sample holders or the bio-hazard bags, the processing point physically inspects and confirms with the list whether all the bio-medical sample holders are received or not. If all the bio-medical sample holders are not received the processing point informs the collection point and asking for resending, resulting in delay. Also, it is difficult to verify the bio-medical sample holders when packed in the biohazard bags unless the biohazard bags are opened. Thus, it is observed that recording of each of the bio-medical sample holders, number of bio-medical sample holders packaged into one biohazard bag and other information are logged manually by many individuals at the collection point and are passed through many hands resulting in wrong labelling, miscommunication, misplacements or missing of bio-medical sample holders.

The processing point is also not aware in advance the number of bio-medical samples which are to be received and with arrival time and the location of the collection point. Hence, management of resources of labors, machines or apparatuses and test materials is challenging.

Another challenge is making availability of bio-medical sample holders at the collection point. At present, there is no track on outgoing bio-medical sample holders and hence the usage is not known.

Tracking of bio-medical samples are known in the prior art, for example from patent document US2003120633A1 in which a clerk is required to read/scan the barcode into the system and requires manual intervene at each station for reading and tracking.

None of the known systems and methods for bio-medical samples identification and resource optimization facilitates continuous reading or scanning of multiple bio-medical samples without manual intervenes of one or multiple bio-medical sample holders at one instant. Further, the known systems and methods of tracking all bio-medical samples holders held in one or more bio-hazard bag at one instant is challenging. A system and method for bio-medical samples identification and resource optimization is in need that eliminates need of manual intervene for reading or scanning, does continuous reading or scanning of multiple bio-medical sample holders and resource management of bio-medical sample holders and resources at a processing point.

### OBJECTS OF THE INVENTION

Some of the objects of the arrangement of the present disclosure are aimed to ameliorate one or more problems of the prior art or to at least provide a useful alternative and are listed herein below.

A principle object of the present disclosure is to provide a system and method for bio-medical samples identification and resource optimization that in real-time is capable to continuously read and record, without manual intervene, multiple bio-medical sample holders, individually or in groups and provides effective recording, communication and resource management of bio-medical sample holders and resource management at a processing point.

Another object of the present disclosure is to provide a system and method for bio-medical samples identification and resource optimization that eliminates the need of manual intervenes at a collection point for reading or scanning bio-medical sample holders thus saves energy, time and cost associated therewith.

Still another object of the present disclosure is to provide a system and method for bio-medical samples identification and resource optimization that maintains an easily accessible database, verifies data and provides data for planning workflow management.

Yet another object of the present disclosure is to provide a system and method for bio-medical samples identification and resource optimization that reads or scans multiple bio-medical sample holders positioned individually or in a bio-hazard bag at one instant.

Another object of the present disclosure is to provide a system and method for bio-medical samples identification and resource optimization that provides effective communication with reduced manpower and hence avoid misplacement and misidentification and facilitates quick turnover time.

Other objects and advantages of the present disclosure will be more apparent from the following description when read in conjunction with the accompanying figures, which are not intended to limit the scope of the present disclosure.

### SUMMARY OF THE INVENTION

The system for bio-medical samples identification and resource optimization at a collection point of the present invention has advantage of operating in real-time environment to continuously scan and record, without manual intervene, multiple bio-medical sample holders, individually or in groups and provides effective recording, communication and resource management of bio-medical sample holders. The system for bio-medical samples identification and resource optimization at a collection point includes a cloud computing network, a plurality of bio-medical sample tagged holders and an outgoing mail pick-up bin. The cloud computing network includes a cloud storage, a cloud processor which is in communication with the cloud storage and a communication platform. The communication platform establishes communication connection between a collection-communication device which is provided at the collection point, the cloud storage and the cloud processor. The plurality of bio-medical sample tagged holders is available at the collection point. Each bio-medical sample tagged holder (30) is defined with a bio-medical sample holder which is tagged with a unique RFID tag and each bio-medical sample holder is filled with bio-medical sample of a person undergoing bio-medical examination. Filled bio-medical sample tagged holders filled with bio-medical sample tagged holders are accumulated at the collection point.

The outgoing mail pick-up bin is disposed at the collection point. The outgoing mail pick-up bin has a cavity to receive accumulated bio-medical sample tagged holders. The outgoing mail pick-up bin is defined with a housing, a RFID reader and a RFID communication device. The housing is positioned inside the cavity. The RFID reader is defined with one or more antenna that transmits electromagnetic waves and triggers unique RFID tags for transmitting data in form of radio waves to the RFID reader for decoding in one or more digital data. The RFID communication device is in connection with the RFID reader and displays the read digital data of the RFID reader. The RFID communication device is in communication with the communication platform to store read digital data in the cloud storage. The RFID communication device displays read digital data. The collection-communication device, through the communication platform, accesses read digital data stored in the cloud storage and link with respective person information at the collection point. The communication platform notifies read digital data to a processing-communication device at a processing point for arrival of expected bio-medical samples at a predetermined time for processing and thereby optimizes resources.

In additional embodiment, a delivery partner is included in the system having a partner communication device, featured with the communication platform, for receiving pick-up intimation, generated by the RFID communication device at the collection point.

In one embodiment, the outgoing mail pick-up bin is defined with at least one indicator that indicates receipt of bio-medical sample tagged holders, scanning of bio-medical sample tagged holders and functional error of the RFID reader and the RFID communication device.

Typically, read digital data of dropped and removed bio-medical sample tagged holders in the outgoing mail pick-up bin is recorded and displayed in a predefined time frame in the RFID communication device and storing in the cloud storage.

In additional embodiment, the manufacturing unit of bio-medical sample tagged holders is notified by the communication platform (23), the utilized number of bio-medical sample tagged holders for replenishment requirement at the collection point.

The present disclosure also discloses a method for bio-medical samples identification and resource optimization at a collection point. The method includes:
providing a cloud computing network is defined with a cloud storage, a cloud processor in communication with the cloud storage and a communication platform;
establishing connection between the cloud storage, the cloud processor, a collection-communication device at the collection point through the communication platform;
introducing bio-medical samples, of persons undergoing medical examination at the collection point, in bio-medical sample tagged holders available at the collection point, wherein each bio-medical sample tagged holder is defined with a bio-medical sample holder tagged with a unique RFID tag, wherein each bio-medical sample holder is defined with a unique RFID tag;
accumulating filled bio-medical sample tagged holders at the collection point; dropping bio-medical sample holders in an outgoing mail pick-up bin which is disposed at the collection point, wherein the outgoing mail pick-up bin having a cavity which is defined with a housing for a RFID reader and a RFID communication device, the outgoing mail pick-up bin executes:
   scanning continuously unique RFID tags of bio-medical sample tagged holders which are dropped in the cavity, wherein, the RFID reader is defined with one or more antennas that transmit electromagnetic waves and triggers unique RFID tags to transmit data in form of radio waves to the RFID reader for decoding in one or more digital data;
   displaying the read digital data of the RFID reader on a RFID communication device;
   storing the read digital data in the cloud storage by communication between the RFID communication device to the communication platform;
   accessing by the collection-communication device, through the communication platform reads digital data stored in the cloud storage and link with a respective person information at the collection point,
   notifying, through the communication platform, read digital data to a processing-communication device at a processing point for arrival of expected bio-medical samples at a predetermined time for processing and thereby optimize resources.

In additional embodiment, the step includes generating request for pick-up, by the RFID communication device at the collection point, to a delivery partner on a partner communication device, featured with the communication platform.

### BRIEF DESCRIPTION OF ACCOMPANYING DRAWINGS

The present disclosure will now be described with the help of the accompanying drawings, in which:

FIG. 1 illustrates a schematic representation of a system (100) for bio-medical samples (07) identification and resource optimization, in accordance with one embodiment of the present disclosure, which includes a cloud network (20) with a cloud database (21), a cloud processor (22) and a communication platform (23), a plurality of bio-medical sample tagged holders (30) each having a bio-medical sample holder (31) to be tagged with a unique RFID tag (32) and an outgoing mail pick-up bin (40);

FIG. 2 illustrates schematic representation of an outgoing mail pick-up bin (40) of the FIG. 1 to be used at the collection point (10);

FIG. 3 illustrates a schematic exploded view of the outgoing mail pick-up bin (40) of the FIGS. 1 and 2;

FIG. 4 illustrates a schematic representation of the outgoing mail pick-up bin (40) of the FIG. 1 reading a bio-medical sample tagged holders (30) positioned in a bio-degradable bag (33) and displaying read information;

FIG. 5 illustrates another schematic representation of the read results of the outgoing mail pick-up bin (40);

FIG. 6 illustrates a schematic representation of a typical RFID tag (32) read by a RFID reader (43) of the outgoing mail pick-up bin (40);

FIG. 7 illustrates a flowchart representing communication between the cloud network (20) facilitating communication of the collection-communication device (11), the processing-communication device (51) and RFID communication device (44);

FIG. 8 illustrates a flowchart representing the method for bio-medical samples (07) identification and resource optimization;

FIG. 9 illustrates a flowchart of the method performed at the processing unit (50);

FIG. 10 illustrates a flowchart of the method performed at the outgoing mail pick-up bin (40); and

FIG. 11 illustrates a flowchart of the method of providing indication by the outgoing mail pick-up bin (40).

### DETAILED DESCRIPTION OF THE INVENTION

In general, as will be understood by one of ordinary skill in the art of knowing systems and method for bio-medical samples identification and resource optimization utilized at a collection point and delivered to a processing point, instead of utilizing manpower for manually recording data, which causes wrong labeling, mishandling, misplacing, misidentification, unmanaged resources and more consumption of time, or system and method for identification and resource optimization of bio-medical samples, which utilizes manually scanned barcode for recording which is time consuming, the system and method claimed herein eliminates the need for manual intervene for scanning, as described herein. There is facility of scanning and recording a number of bio-medical samples with no or minimal manual intervene and results in avoiding errors caused by humans and also facilitates resource optimization based on real-time conditions such as quantity, location, type of tests and the like.

Moreover, in the present disclosure, rather than having manual intervenes for recording bio-medical samples, the recording is achieved by using an outgoing mail pick-up bin which continuously scans one or multiple bio-medical sample tagged holders filled with bio-medical samples and provides digitalized data which is used for resource optimization.

Further, the invention, allows planning of workflow at the processing point based on the collection quantity of bio-medical samples communicated by the communication point or reroute bio-medical sample holders in event of wrong delivery. Also, the invention, make availability of bio-medical sample tagged holders at the collection point by tracking the outgoing bio-medical sample tagged holders from the collection point.

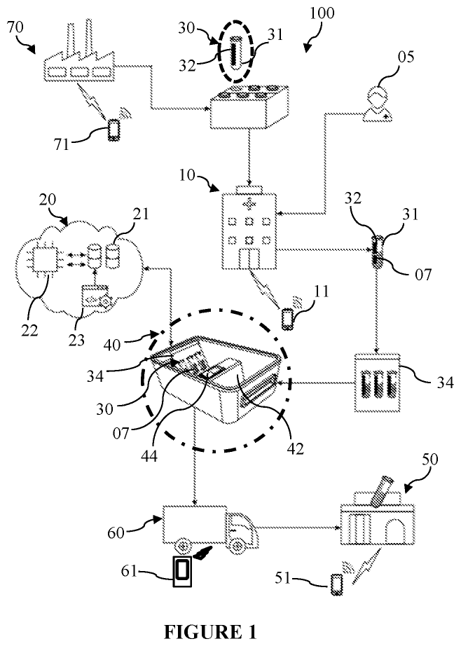

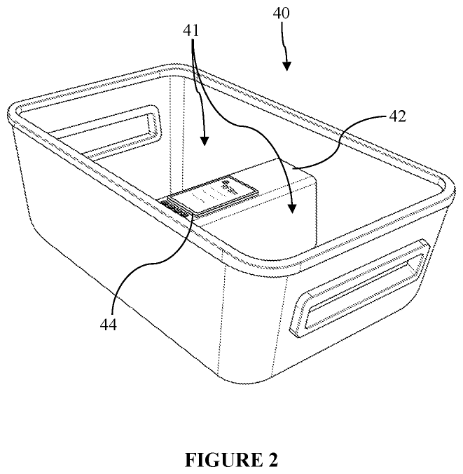

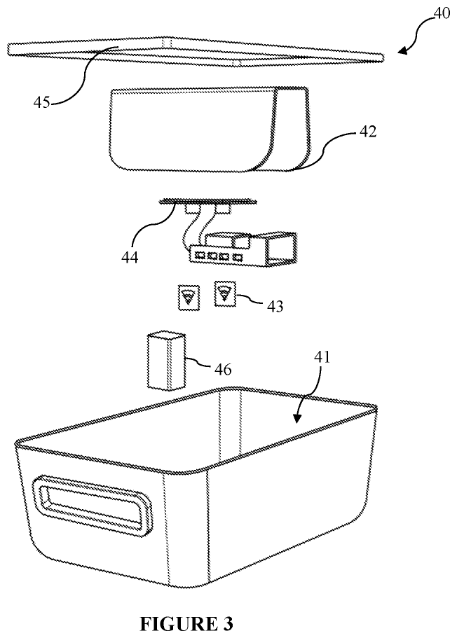

Referring now to the drawings, FIGS. 1 to 11, where the present invention is generally referred to with numeral (100), it can be observed that a system for bio-medical samples (07) identification and resource optimization, in accordance with an embodiment, is provided which includes a cloud computing network (20), a plurality of bio-medical sample tagged holders (30) and an outgoing mail pick-up bin (40).

Herein, the present disclosure, a collection point (10) is the point from where the bio-medical samples are collected from the persons undergoing bio-medical examination. For example, the collection point (10) can be a hospital, a clinic, a residential or commercial unit and the like. A processing point (50) is a point where the bio-medical samples are delivered for processing like examination and report generation. For example, the processing point (50) can be a laboratory or an examination center. A manufacturing unit (70) is a unit where bio-medical sample tagged holders (30) are manufactured and delivered to the collection point (10) which are utilized for introducing bio-medical samples (07). A delivery partner (60) is a partner that transports bio-medical sample tagged holders (30) filled with bio-medical samples from the collection point (10) to the processing point (50).

The collection point (10) has one or more collection-communication device (11) for uploading and accessing information and data. The processing point (50) has one or more processing-communication device (51). The collection-communication device (11) and the processing-communication device (51) are required to have internet connectivity for communication with the cloud computing network (20).

In the present disclosure, the collection point (10) and the processing point (50) are not limited to one and can include more than one. For example, there can be one collection point (10) and multiple processing points (50), multiple collection points (10) and one processing point (50), multiple collection points (10) and multiple processing points (50) or one collection point (10) to one processing point (50). For clear understanding of the present invention, the exemplary embodiment described herein, is by considering tracking of bio-medical samples (07) from one collection point (10) to one processing point (50). However, the number of collection point (10) and the number of processing point (50) can be added by connecting the collection-communication device (11) for each collection point (10) and by connecting the processing-communication device (51) for each processing point (50).

The cloud computing network (20) includes a cloud storage (21), a cloud processor (22) and a communication platform (23). The cloud storage (21) stores digital data of the system (100). The cloud processor (22) is in communication with the cloud storage (21). The cloud processor (22) processes information received in the system (100) and the working is described in subsequent paragraphs. The communication platform (23) is a platform that is installed in the collection-communication device (11) and the processing-communication device (51) and facilitates communication between the collection-communication device (11), the processing-communication device (51) with the cloud storage (21) and the cloud processor (22).

The plurality of bio-medical sample tagged holders (30) is available at the collection point (10). Each bio-medical sample tagged holder (30) is defined with a bio-medical sample holder (31) which is tagged with a unique RFID tag (32) and each bio-medical sample holder (31) is filled with bio-medical sample of a person (05) undergoing bio-medical examination.

Filled bio-medical sample tagged holders (30) are accumulated at the collection point (10). In one embodiment, a number of filled bio-medical sample tagged holders (30) are packed in a packaging bag or a biohazard bag (34). For example, two to three different bio-medical samples (07) of a person (05) is packed in one biohazard bag (34).

In one embodiment, the plurality of bio-medical sample tagged holders (30) is made available to the collection point (10) by the manufacturing unit (70). The manufacturing unit (70) makes assembly of bio-medical sample holder (31) with the unique RFID tag (32) and delivers to the collection point (10) so that the collection point (10) do not need to invest and manage manpower to perform tagging rather can focus more on the core activities of collection and dispatch. In another embodiment, the assembly of the bio-medical sample holder (31) and the unique RFID tag (32) is performed at the collection point (10). In yet another embodiment, the assembly of the bio-medical sample holders (31) and unique RFID tags (32) is performed at the processing point (50) and then supplied to the collection point (10). Though, the present disclosure discloses that the assembly of the bio-medical sample holders (31) and unique RFID tags (32) can be performed at the collection point (10) or the processing point (50), it is preferred that the assembly of the bio-medical sample holders (31) and unique RFID tags (32) is performed at the manufacturing unit (70) as the manufacturing unit (70) can create a digital record of type and quantity of assembled bio-medical sample tagged holders (30) and which are to be supplied to the collection point (10) and the digital record are shared by a manufacturing-communication device (71) to the communication platform (23) which can be accessed by the collection-communication device (11) and hence the collection point (10) does not require digitalization of the data and saves on manpower work and cost associated therewith.

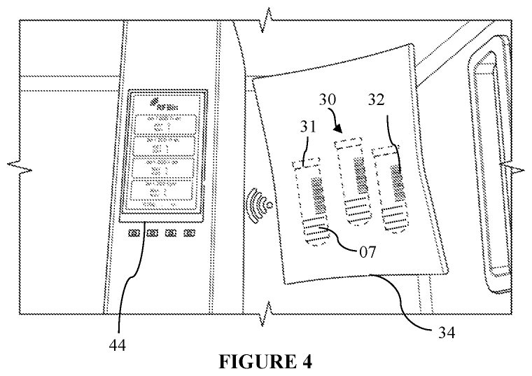

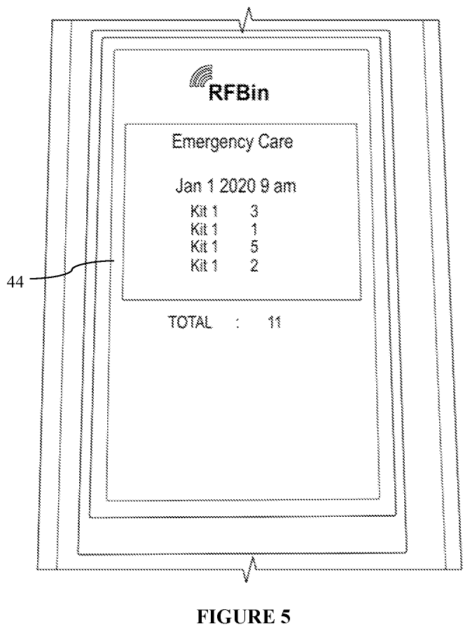

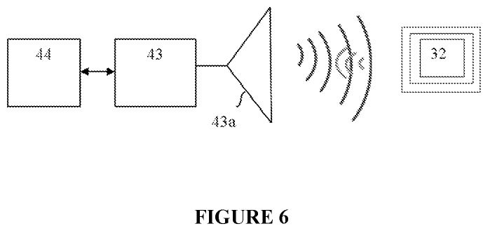

The outgoing mail pick-up bin (40) is disposed at the outgoing section of the collection point (10). The outgoing mail pick-up bin (10) has a cavity (41) which receives accumulated bio-medical sample tagged holders (30) which are collected with bio-medical sample (07). The outgoing mail pick-up bin (40) is defined with a housing (42), a RFID reader (43) and a RFID communication device (44). In one embodiment, the housing (42) is disposed in the cavity (41). The RFID reader (43) is disposed in the housing (42). The RFID reader (43), in operative configuration, continuously scans unique RFID tags (32) of bio-medical sample tagged holders (30) which are dropped in the cavity (41). The RFID reader (43) scans RFID tags (32) irrespective of bio-medical sample tagged holders (30) are positioned directly in the cavity (41) or placed in the bio-hazard bag (34). Thus, without the need to open the bio-hazard bag (34), scanning of the RFID tags (32) is performed. Also, the RFID reader (43) scans all the RFID tags (32) dropped in the cavity (41) at an instant and hence scanning is performed in faster time as compared to manual scanning or manual recording. The RFID reader (43) is defined with one or more antenna (43a) that transmits electromagnetic waves and triggers unique RFID tags (32) to transmit data in form of radio waves to the RFID reader (43) for decoding in one or more digital data. The RFID communication device (44) is in connection with the RFID reader (43) and displays the read digital data of the RFID reader (43). The RFID communication device (44) is in communication with the communication platform (23) to store read digital data in the cloud storage (21). The RFID communication device (44) displays read digital data. In one embodiment, read digital data of dropped and removed bio-medical sample tagged holders (30) in the cavity (41) is recorded and displayed in a predefined time frame on the RFID communication device (44) and stored in the cloud storage (21) along with the predefined time frame. For example, every 24 hours read digital data are recorded and stored in the cloud storage (21) corresponding to the date and time as exemplarily shown in FIGS. 4 and 5.

Further, RFID communication device (44) stores in the cloud storage (21) the number of bio-medical sample tagged holders (30) dropped in and removed from the cavity (41), thus knowing the count for inventory management. In an additional embodiment, the outgoing mail pick-up bin (40) is defined with at least one indicator (45) that indicates receipt of bio-medical sample tagged holders (30), scanning of bio-medical sample tagged holders (30) and functional error of said RFID reader (43) and the RFID communication device (44). In event when the indicator (45) is a light indicator, then green colored light indicates the receipt of the receipt of bio-medical sample tagged holders (30) in the cavity (41), orange colored light indicates the scanning is performed and the red colored light indicates that there is some functional error, for example the scanning is not performed or the scanned data is not communicated to the RFID communication device (44) and the like system errors. The color of the light can be set as required and is not limited to the once mentioned in the present disclosure. In one embodiment, a power storage battery (46) is provided to power the components of the outgoing mail pick-up bin (40).

The collection-communication device (11), through the communication platform (23), accesses read digital data stored in the cloud storage (21) and link with respective person information at the collection point (10). The personal information can be manually filled in the communication platform (23) or can be linked with a database created at the collection point (10).

The communication platform (23) notifies read digital data to the processing-communication device (51) of the processing point (50) to expect arrival of bio-medical samples (07) at a predetermined time for processing and thereby optimize resources like manpower, machineries or materials for conducting examination. As the resources are optimized, there is quick turnover, thus saving on the processing time. The predetermined time can be a day before the arrival of bio-medical samples (07). For example, as soon as the outgoing mail pick-up bin (40) scans the accumulated bio-medical sample tagged holders (30), the number of the accumulated bio-medical sample tagged holders (30) is stored in the cloud storage and the notification is sent to the processing point (50) to expect the arrival and the number of accumulated bio-medical sample tagged holders (30) from the collection point (10). Also, the location of the collection point (10) is known so the processing point (50) is notified with the number of accumulated bio-medical sample tagged holders (30) and location of arrival.

In one additional embodiment, in event when the collection point (10) is at a distance from the processing unit (50), then the delivery partner (60) is required to carry accumulated bio-medical sample tagged holders (30) from the collection point (10) to the processing point (50). The delivery partner (60) has a partner communication device (61) which is featured with the communication platform (23). The communication platform (23) when receives information from the outgoing mail pick-up bin (10) of receipt of accumulated bio-medical sample tagged holders (30), the communication platform (23) signals the delivery partner (60) for pick-up of accumulated bio-medical sample tagged holders (30) from the collection point (10) and deliver to the processing point (50). Thus, there is no manual intervene.

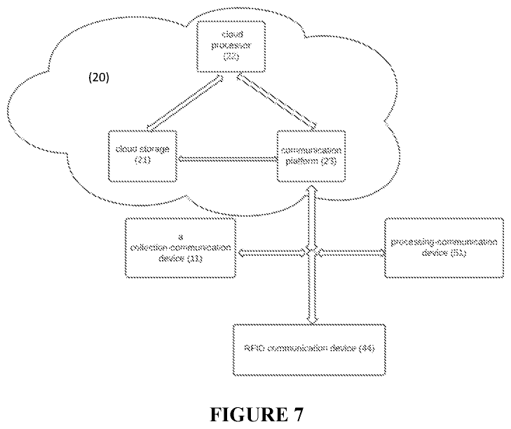

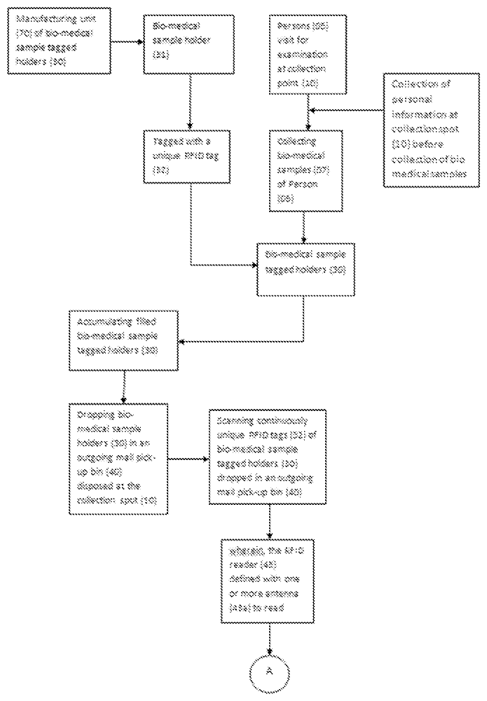

The present disclosure also discloses a method for bio-medical samples (07) identification and resource optimization. The best-method includes providing the cloud computing network (20) which includes the cloud storage (21), the cloud processor (22) and the communication platform (23) and establishing connection between the cloud storage (21), the cloud processor (22), the collection-communication device (11) at the collection point (10), the processing-communication device at the processing point (50) and the RFID communication device (44) through the communication platform (23) as illustrated in FIG. 7.

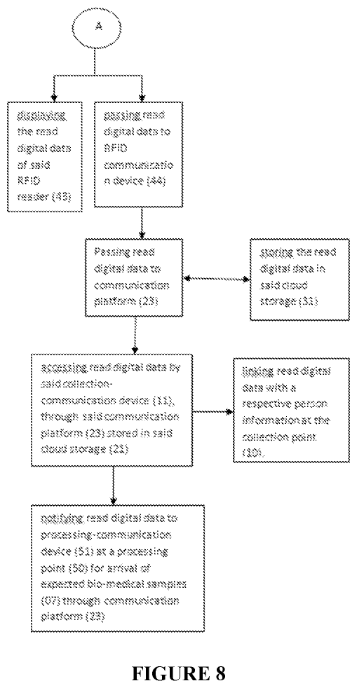

As illustrated in FIG. 8, in one embodiment, the manufacturing unit (70) manufactures bio-medical sample tagged holders (30) by tagging unique RFID tags (32) with bio-medical sample holders (31) and supplying the bio-medical sample tagged holders (30) to the collection point (10) and also provides digitalized data list of the bio-medical sample tagged holders (30) to the collection point (10) through the communication platform (23). The collection point (10) fills bio-medical samples (07) of persons (05) undergoing bio-medical examination into the received bio-medical sample tagged holders (30) and accumulates all the filled bio-medical sample tagged holders (30). In one embodiment, the accumulated bio-medical sample tagged holders (30) are introduced in the biohazard bag (34). The accumulated bio-medical sample tagged holders (30) are dropped in the cavity (41) of the outgoing mail pick-up bin (40). In the outgoing mail pick-up bin (40), continuous scanning of unique RFID tags (32) of bio-medical sample tagged holders (30) which are dropped in the cavity (41) is performed by the RFID reader (43). The RFID reader (43), as disclosed, is defined with one or more antenna (43a) that transmits electromagnetic waves and triggers unique RFID tags (32) to transmit data in form of radio waves to the RFID reader (43) for decoding in one or more digital data. Displaying the read digital data of the RFID reader (43) on the RFID communication device (44) and know the number of bio-medical sample tagged holders (30) which are going out from the collection point (10). The read digital data is stored in the cloud storage (31) by communication between the RFID communication device (44) to the communication platform (23). The collection-communication device (11), through the communication platform (23) accesses read digital data stored in the cloud storage (21) and links with a respective person information, such as name, age, gender, date of birth, type of examination required and the like, at the collection point (10). The next step is notifying, through the communication platform (23) read digital data to the processing-communication device (51) at the processing point (50) for arrival of expected bio-medical samples (07) at the predetermined time for processing, location of the collection point (10) and thereby assist in optimizing resources at the processing point (50).

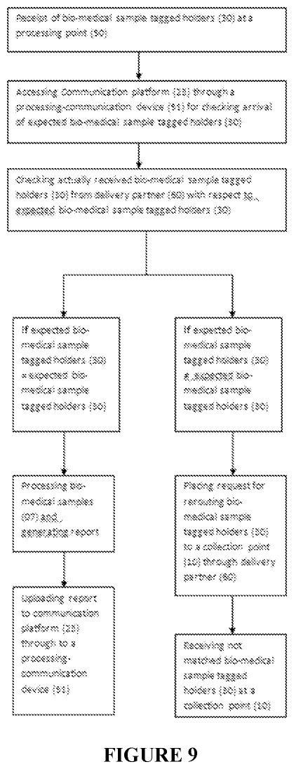

As illustrated in FIG. 9, bio-medical sample tagged holders (30) are received at the processing point (50) and upon receipt accessing the communication platform (23) through the processing-communication device (51) for checking arrival of expected bio-medical sample tagged holders (30). Checking actually received bio-medical sample tagged holders (30) from the delivery partner (60) with respect to expected bio-medical sample tagged holders (30). In event when expected bio-medical sample tagged holders (30) is equal to the expected bio-medical sample tagged holders (30), the bio-medical samples (07) are processed and the report is generated which is uploaded to the cloud storage (21) to be accessed by the collection point (10) and/or the person (05) to whom the report belongs. In event when the expected bio-medical sample tagged holders (30) is not equal to the expected bio-medical sample tagged holders (30), then a request for rerouting of bio-medical sample tagged holders (30) to a collection point (10) through the delivery partner (60) is placed and hence requires no manual intervene. Receiving at the collection point (30) rerouted bio-medical sample tagged holders (30).

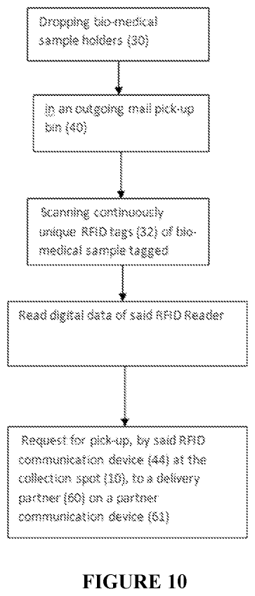

In one embodiment, as illustrated in FIG. 10, dropping bio-medical sample holders (30) in the outgoing mail pick-up bin (40) where continuous scanning of unique RFID tags (32) is performed by the RFID reader (43). Once scanning is done, the request for pick-up, is issued to the delivery partner (60) on the partner communication device (61) by the RFID communication device (44).

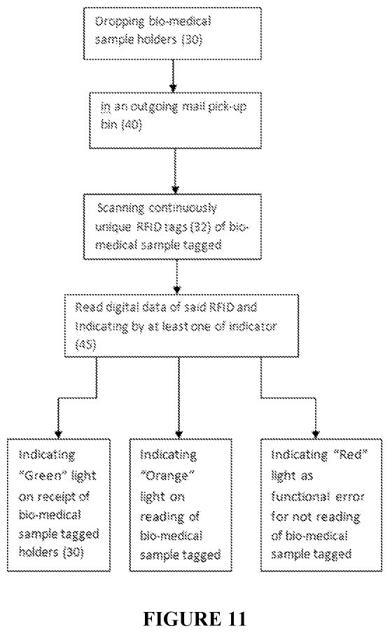

In one embodiment, as illustrated in FIG. 11, bio-medical sample tagged holders (30) are dropped in the outgoing mail pick-up bin (40) in which continuous scanning of unique RFID tags (32) of bio-medical sample tagged holders (30) is performed. The indicator (45) indicates receipt of bio-medical sample tagged holders (30), scanning of bio-medical sample tagged holders (30) and functional error of said RFID reader (43) and the RFID communication device (44). In event when the indicator (45) is a light indicator, then green colored light indicates the receipt of the receipt of bio-medical sample tagged holders (30) in the cavity (41), orange colored light indicates the scanning is performed and the red colored light indicates that there is some functional error, for example the scanning is not performed or the scanned data is not communicated to the RFID communication device (44) and the like system errors.

The method additionally includes notifying the manufacturing-communication device (71) of the manufacturing unit (70), through the communication platform (23), the requirement of replenishment of bio-medical sample tagged holders (30) at the collection point (10).

The foregoing description conveys the best understanding of the objectives and advantages of the present invention. Different embodiments, steps or alternatives may be made of the inventive concept of this invention. It is to be understood that all matter disclosed herein is to be interpreted merely as illustrative, and not in a limiting sense.

What is claimed is:

1. A system for bio-medical samples identification and resource optimization, said system comprising:
a cloud computing network defined with:
a cloud storage;
a cloud processor in communication with said cloud storage; and
a communication platform to establish communication connection between:
a collection-communication device at a collection point;
said cloud storage; and
said cloud processor;
a plurality of bio-medical sample tagged holders available at the collection point, each bio-medical sample tagged holder defined with a bio-medical sample holder tagged with a unique RFID tag and each bio-medical sample holder filled with bio-medical sample of a person undergoing bio-medical examination, filled bio-medical sample tagged holders accumulated at the collection point; and
an outgoing mail pick-up bin disposed at the collection point, said outgoing mail pick-up bin having a cavity configured to receive accumulated bio-medical sample tagged holders, said outgoing mail pick-up bin defined with:
a housing positioned inside the cavity;
a RFID reader disposed in said housing and in operative configuration continuously scan unique RFID tags of bio-medical sample tagged holders dropped in the cavity, said RFID reader defined with one or more antenna that transmit electromagnetic waves and triggers unique RFID tags to transmit data in form of radio waves to said RFID reader for decoding in one or more digital data; and
a RFID communication device in connection with said RFID reader and configured to display the read digital data of said RFID reader, said RFID communication device in communication with said communication platform to store read digital data in said cloud storage, said RFID communication device configured to display read digital data;
wherein, said collection-communication device, through said communication platform, accesses read digital data stored in said cloud storage and link with respective person information at the collection point,
wherein, said communication platform notifies read digital data to a processing-communication device at a processing point for arrival of expected bio-medical samples at a predetermined time for processing and thereby optimize resources.

2. The system for bio-medical samples identification and resource optimization as claimed in claim 1, include a delivery partner having a partner communication device, featured with the communication platform, for receiving pick-up intimation, generated by said RFID communication device at the collection point.

3. The system for bio-medical samples identification and resource optimization as claimed in claim 1, wherein said outgoing mail pick-up bin is defined with at least one indicator that indicates receipt of bio-medical sample tagged holders, scanning of bio-medical sample tagged holders and functional error of said RFID reader and said RFID communication device.

4. The system for bio-medical samples identification and resource optimization as claimed in claim 1, wherein read digital data of dropped and removed bio-medical sample tagged holders is recorded and displayed in a predefined time frame in the RFID communication device and storing in said cloud storage.

5. The system for bio-medical samples identification and resource optimization as claimed in claim 1, wherein a manufacturing unit of bio-medical sample tagged holders notified by said communication platform, the utilized number of bio-medical sample tagged holders for replenishment requirement at the collection point.

6. A method for bio-medical samples identification and resource optimization comprising:
providing a cloud computing network defined with a cloud storage, a cloud processor in communication with said cloud storage and a communication platform;
establishing connection between said cloud storage, said cloud processor, a collection-communication device at the collection point through said communication platform;
introducing bio-medical samples, of persons undergoing medical examination at the collection point, in bio-medical sample tagged holders available at the collection point, wherein each bio-medical sample tagged holder defined with a bio-medical sample holder tagged with a unique RFID tag, wherein each bio-medical sample holder is defined with a unique RFID tag;
accumulating filled bio-medical sample tagged holders at the collection point; and
dropping bio-medical sample tagged holder in an outgoing mail pick-up bin disposed at the collection point, wherein said outgoing mail pick-up bin having a cavity defined with a housing for a RFID reader and a RFID communication device, said outgoing mail pick-up bin execute:
scanning continuously unique RFID tags of bio-medical sample tagged holders dropped in the cavity,
wherein, said RFID reader defined with one or more antenna that transmit electromagnetic waves and triggers unique RFID tags to transmit data in form of radio waves to said RFID reader for decoding in one or more digital data;
displaying the read digital data of said RFID reader on a RFID communication device;
storing the read digital data in said cloud storage by communication between said RFID communication device to said communication platform;
accessing by said collection-communication device, through said communication platform read digital data stored in said cloud storage and link with a respective person information at the collection point; and
notifying, through said communication platform read digital data to a processing-communication device at a processing point for arrival of expected bio-medical samples at a predetermined time for processing and thereby optimize resources.

7. The method for bio-medical samples identification and resource optimization as claimed in claim 6, wherein the step includes generating request for pick-up, by said RFID communication device at the collection point, to a delivery partner on a partner communication device, featured with the communication platform.

8. The method for bio-medical samples identification and resource optimization as claimed in claim 6, wherein indicating by at least one indicator defined with said outgoing mail pick-up bin the receipt of bio-medical sample tagged holders, scanning of bio-medical sample tagged holders and functional error of said RFID reader and said RFID communication device.

9. The method for bio-medical samples identification and resource optimization as claimed in claim 6, wherein recording and displaying in a predefined time frame dropped and removed bio-medical sample tagged holders in the RFID communication device and storing in said cloud storage.

10. The method for bio-medical samples identification and resource optimization as claimed in claim 6, notifying a manufacturing unit, through said communication platform, requirement of replenishment of bio-medical sample tagged holders at the collection point.
